(19) United States
(12) Patent Application Publication
(10) Pub. No.: US 2024/0370902 A1
(43) Pub. Date: Nov. 7, 2024

(54) ARTIFICIAL INTELLIGENCE AND MACHINE LEARNING-ENHANCED CUSTOMIZABLE PATIENT COMMUNICATIONS PLATFORM

(71) Applicant: Robert Miglani, Monroe Township, NJ (US)

(72) Inventor: Robert Miglani, Monroe Township, NJ (US)

(21) Appl. No.: 18/653,873

(22) Filed: May 2, 2024

Related U.S. Application Data
(60) Provisional application No. 63/463,473, filed on May 2, 2023.
(60) Provisional application No. 63/463,475, filed on May 2, 2023.

(51) Int. Cl. G06Q 30/0251; G06Q 30/0241; G06Q 30/0242; G16H 40/00

(52) U.S. Cl. CPC G06Q 30/0271; G06Q 30/0244; G06Q 30/0276; G16H 40/00

(57) ABSTRACT

The present invention relates to a personalized marketing and communication system for healthcare practices that incorporates artificial intelligence (AI) and machine learning (ML) technologies to optimize marketing campaigns, automate lead scoring and nurturing, perform predictive analytics on patient behavior and trends. The system configured to utilize AI and ML algorithms for personalization of marketing campaigns, automated lead scoring and nurturing, and predictive analytics. The system delivers personalized communications via preferred channels and provides customizable content suggestions and tools based on AI and ML algorithms. The system tracks various performance metrics and optimize marketing channels and identifies correlations, patterns, and trends within the patient data using AI and machine learning techniques.

### CROSS-REFERENCE TO RELATED APPLICATION

This application claims priority benefit of U.S. Provisional Patent Application No. 63/463,473 filed on May 2, 2024 entitled “Customizable System for Managing Personalized Patient Communications in Healthcare Practices,” and U.S. Provisional Patent Application No. 63/463,473 filed on May 2, 2024 entitled “Artificial Intelligence and Machine Learning-Enhanced Customizable Marketing Automation Platform for Personalized Patient Communications in Healthcare Practices,” the disclosure of which are incorporated herein by reference in their entirety.

### BACKGROUND OF THE INVENTION

### 1. Field of the Disclosure

The present disclosure is generally related to marketing automation platforms, and more particularly, to a customizable marketing automation platform designed for small private medical practices, such as eye care, dermatology, physical therapy, and other healthcare domains.

### 2. Description of the Related

The marketing and communication needs of small private medical practices, including eye care practitioners, physical therapists, dermatologists, and others, have remained largely unaddressed, as existing marketing automation platforms have either been too generic or too expensive for these practices to adopt. Although applications of AI in marketing automation exist, none have been tailored specifically to accommodate the unique requirements of private healthcare practices, such as incorporating clinical patient data and adapting to the numerous stages of a patient's healthcare journey across various domains of care.

For instance, myopia care, a critical service provided by eye care professionals to children referred by general practitioners, is a prime example of a domain where effective communication with patients and their parents or guardians is essential. Despite the prevalence and potential long-term consequences of myopia, many eye care professionals' websites lack comprehensive information on its causes and treatments. Moreover, these professionals often struggle to communicate the importance of myopia care in an engaging and informative manner, resulting in patients either conducting their own research or neglecting proper treatment due to the absence of clear, professional, and compelling content.

The demand for a customized marketing automation platform extends beyond myopia care, encompassing various other private healthcare practices such as dry eye care, dermatology, physical therapy, nursing homes, mental health and addiction treatment, birth centers, and more. In each of these domains, the challenge lies in effectively communicating with patients and crafting targeted campaigns that enhance patient engagement and retention.

Therefore, a pressing need exists for a marketing automation platform that can be adapted to the specific needs and drivers of each private healthcare practice. By providing content and communications tailored to the domain of care, this platform would enable healthcare practices to create and manage personalized marketing campaigns and patient communications, leading to improved patient outcomes and overall healthcare quality. Notably, no current AI-driven marketing automation solutions have been specifically designed to address the unique requirements of private healthcare practices, presenting a significant opportunity for innovation in this area.

### SUMMARY OF THE CLAIMED INVENTION

Embodiments of the present invention include systems and methods for personalized marketing and communication in relation to healthcare practices. Personalization may be based on artificial intelligence (AI) and machine learning (ML) technologies to tailor and optimize content campaigns, automation of lead scoring and nurturing, and predictive analytics on patient behavior and trends. Custom models may be developed, and personalized content may be generated and delivered to different individual user devices via preferred channels, including customizable content suggestions and tool recommendations based on AI and ML algorithms. The system tracks various performance metrics and optimize marketing channels and identifies correlations, patterns, and trends within the patient data using AI and machine learning

### BRIEF DESCRIPTIONS OF THE DRAWINGS

FIG. 1 illustrates a system for optimizing patient outcomes and business performance in a healthcare marketing automation system, according to an embodiment.

FIG. 2 illustrates a Base Module, according to an embodiment.

FIG. 3 illustrates a User Module, according to an embodiment.

FIG. 4 illustrates a Marketing Automation Module, according to an embodiment.

FIG. 5 illustrates a Content Generation Module, according to an embodiment.

FIG. 6 illustrates an Analytics Module, according to an embodiment.

FIG. 7 illustrates a Correlation Module, according to an embodiment.

FIG. 8 illustrates a Predictive Analytics Module, according to an embodiment.

FIG. 9 illustrates a block diagram of an exemplary system that may be used to implement an embodiment.

FIG. 10 is a flow chart illustrating an exemplary method for delivery of personalized user content.

FIG. 11 is a flow chart illustrating an exemplary method for delivery of personalized user content.

### DETAILED DESCRIPTION

The presently described AI and ML-enhanced customizable system for managing personalized patient communications in healthcare practices can address the distinct marketing and communication challenges encountered by small private medical practices, such as eye care practitioners, physical therapists, dermatologists, and others. By offering a cost-effective and customizable platform tailored to these practices' specific requirements, the system empowers healthcare professionals to create bespoke marketing messages based on each patient's healthcare data utilizing AI and ML algorithms. The system boosts engagement and retention by delivering personalized communications through preferred channels and presenting AI-driven customizable content suggestions and creation tools. The system employs AI techniques to track performance metrics, enabling practitioners to make data-driven decisions and optimize their marketing campaigns for improved patient outcomes. The generative AI techniques utilize machine learning models that takes in various inputs, such as patient demographics, medical history, history of engagement with digital media assets, interactions with chatbot, the content of digital media assets, practitioner data, etc. and process such inputs to parse elements in the digital media assets and patient data, and identify correlations, patterns, or trends in the data. The inputs to the machine learning models may include previously generated outputs in a feedback loop to improve the accuracy of the model. The machine learning models may generate various outputs such as lead scores, personalized content for engagement, predictions in future engagements, or recommendations for content customization generation and delivery options. The artificial intelligence system may track the actual engagement to the delivered digital media asset and continuously and instantly update the machine learning model based on the actual engagement pattern and any new input to reflect the changes in the trend.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples. The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

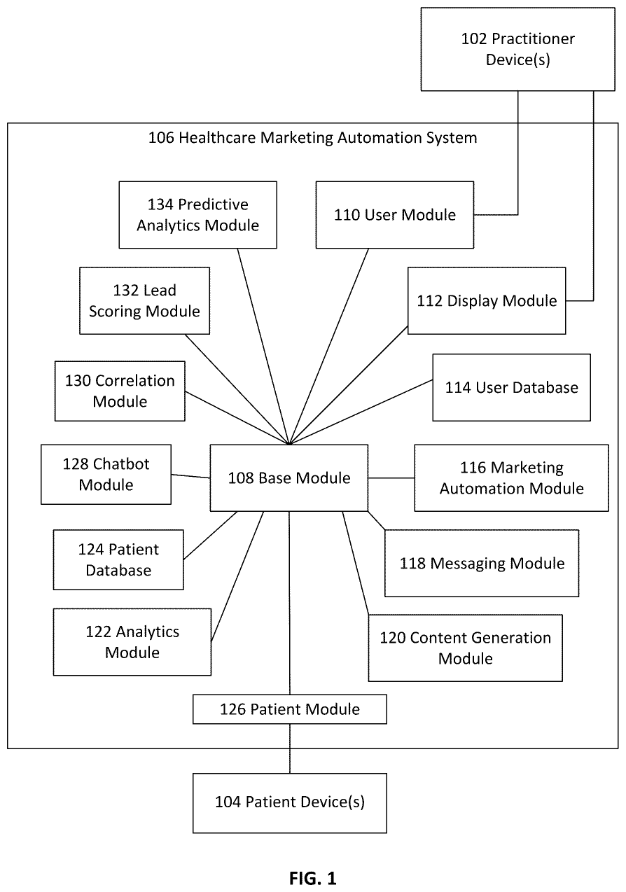

FIG. 1 illustrates a system for optimizing patient outcomes and business performance in a healthcare marketing automation system, according to an embodiment. The system includes Practitioner Device(s) 102, which are electronic devices used by healthcare professionals to access, input, and manage patient and clinical data in the Healthcare Marketing Automation System and may be, for example, desktop computers, laptop computers, tablet devices, smartphones, and specialized medical equipment with built-in computing capabilities, Patient Device(s) 104 are electronic devices used by patients to receive personalized communications from healthcare practices through the Healthcare Marketing Automation System and may be, for example, smartphones, personal computers, tablet devices, smartwatches, and other wearable devices, Healthcare Marketing Automation System 106, which may be a comprehensive platform designed to facilitate personalized patient communications in healthcare practices by facilitating automated marketing and communication between practitioner devices and patient devices, wherein Healthcare professionals use practitioner devices, such as desktop computers or tablets, to access and input patient and clinical data into the system, the data is then used by the Marketing Automation module 106 to create tailored marketing messages based on each patient's healthcare data, the tailored marketing messages may be used as personalized communications which are sent to patients via the Messaging module 118, which delivers the content through the patient's preferred channels, such as, for example, email or SMS, to the patient devices like smartphones or personal computers. Patients may then engage with the content, schedule appointments, and manage their healthcare information using their devices. Base Module 108 serves as the central hub for the Healthcare Marketing Automation System 106, coordinating various modules and functionalities to deliver a seamless user experience. The Base Module 108 receives user input from practitioners via the User Module 110, allowing them to manage patient data and customize marketing campaigns. The Base Module 108 displays information to the user through Display Module 112, providing an interface for navigation and interaction with the Healthcare Marketing Automation System 106. Base Module 108 is responsible for storing and retrieving user data from the User Database 114, ensuring that healthcare professionals' preferences, settings, and other relevant information are readily accessible.

The Base Module 108 connects with the Marketing Automation Module 116 to create automated, customizable marketing campaigns tailored to the patients' healthcare journey stages, utilizing AI algorithms for personalization. The Messaging Module 118 is executed by the Base Module 108 to send automated, customizable messages to patients using their preferred communication channels. Base Module 108 may execute the Content Generation Module 120, which generates customizable content for marketing campaigns and messages, ensuring that communications are engaging and informative. The Analytics Module 122 is also executed by the Base Module 108 to analyze engagement and other metrics associated with campaigns and messages using AI techniques, providing valuable insights for healthcare professionals to optimize their marketing strategies.

Base Module 108 retrieves and stores patient data from Patient Database 124, allowing practitioners to access and manage essential patient information, such as personal details, clinical data, and appointment history. The Base Module 108 further integrates with the Chatbot Module 128 to provide AI-powered customer service and engagement through automated conversation interfaces, enhancing patient support and interactions.

The User Module 110 is a component of the Healthcare Marketing Automation System 106. It provides healthcare professionals with an interface for accessing and managing system functionalities that incorporate artificial intelligence algorithms. Practitioners log into User Module 110 to access patient information from the Patient Database 124 and update patient data. The User Module 110 allows healthcare professionals to manage marketing campaigns via the Marketing Automation Module 116, streamlining the process of creating and customizing campaigns using AI techniques. The User Module 110 enables practitioners to send messages using the Messaging Module 118, which leverages AI for personalization. The User Module 110 facilitates marketing content generation by executing the Content Generation Module 120, providing optimized content for marketing campaigns and messages through use of artificial intelligence and machine learning algorithms. The User Module 110 allows users to access and view analytics generated by the Analytics Module 122, offering insights for data-driven decision-making based on algorithmic analysis. The Display Module 112 is a component of the Healthcare Marketing Automation System 106, designed to present healthcare professionals with an organized and visually appealing interface for viewing and interacting with various data from the User Database 114 and/or Patient Database 124. It achieves this by displaying the information in one or more dashboards, which allow the user to quickly view and/or modify information relating to patients, campaigns, messages, content, and other relevant aspects. In some respects, Display Module 112 may display information relating to AI algorithms currently in use by the Healthcare Marketing Automation System 106.

The User Database 114 is a component of the Healthcare Marketing Automation System 106, responsible for storing and managing user data associated with healthcare practitioners. This data may include the practitioner's area of practice, location, phone number, whether they are accepting new patients, headshot or other photos, marketing campaign data, marketing content data, analytics data, and more. By maintaining this information and utilizing AI algorithms for data processing, the User Database 114 enables the system to personalize and manage marketing campaigns and communications more effectively, as well as provide valuable insights to the practitioners. The stored data in User Database 114 related to Chatbot Module 128, Correlation Module 130, Lead Scoring Module 132, and Predictive Analytics Module 134 can be accessed and utilized by the healthcare practitioner to optimize their marketing campaigns, improve patient communication, prioritize patient outreach efforts, and make data-driven decisions based on forecasts and insights generated by the Healthcare Marketing Automation System 106. The stored user data in the User Database 114 may include, but is not limited to, the following types: Personal Information, Professional Information, Profile Data, Marketing Campaign Data, Marketing Content Data, Analytics Data, Chatbot Data, Correlation Data, Lead Scoring Data, and Predictive Analytics Data. Personal Information may include data such as the practitioner's name, contact information, and login credentials (e.g., a unique username and password combination). For example, Dr. Jane Smith's name, phone number, email address, and login credentials may be stored in the User Database 114 for secure access to the system. Professional Information may encompass data related to the practitioner's area of practice, location, phone number, and whether they are accepting new patients. For example, Dr. Smith's specialization in pediatrics, her practice's address, contact number, and her current patient acceptance status may be stored in the database. Profile Data may include information such as the practitioner's headshot or other photos, biographical information, and credentials. For example, a professional headshot of Dr. Smith, her educational background, and certifications may be stored in User Database 114 to be displayed on her practice's website. Marketing Campaign Data may comprise information related to the practitioner's ongoing and past marketing campaigns, such as target audience, campaign duration, and promotional materials. For example, Dr. Smith's recent campaign promoting annual check-ups for children, including the start and end dates, target age groups, and the promotional materials used, may be stored in User Database 114. Marketing Content Data may include data related to the marketing content created or used by the practitioner, such as articles, images, videos, and templates. For example, an article written by Dr. Smith on the importance of vaccinations and a series of educational videos she uses in her campaigns may be stored in User Database 114. Analytics Data may include information related to the performance metrics of the practitioner's marketing campaigns, such as engagement, response rates, conversion rates, and retention. For example, the clickthrough rate, the number of appointments scheduled, and the retention rate of patients who participated in Dr. Smith's annual check-up campaign may be stored in User Database 114 for future analysis and campaign optimization. Chatbot Data may include settings, customizations, and conversation logs related to the Chatbot Module 128. For example, Dr. Smith's preferred chatbot greeting, the chatbot's responses to frequently asked questions, and chat logs from patient interactions may be stored in the User Database 114 for ongoing analysis and improvement. Correlation Data may comprise insights and patterns identified by the Correlation Module 130, which can be used to optimize marketing campaigns and patient communication. For example, Dr. Smith's discovery of trends in patient behavior and preferences, as well as the correlations between specific content types and patient engagement, may be stored in the User Database 114. Lead Scoring Data may include the lead scores assigned to patients by the Lead Scoring Module 132, helping the practitioner prioritize and target patients for specific campaigns or follow-up communications. For example, the lead scores of Dr. Smith's patients, indicating their likelihood to engage with marketing campaigns or adhere to treatment plans, may be stored in User Database 114. Predictive Analytics Data may consist of predictions and forecasts generated by the Predictive Analytics Module 134, such as patient behavior predictions, campaign performance forecasts, and healthcare outcome predictions. For example, Dr. Smith's forecasted effectiveness of her flu vaccination campaign, along with patient behavior predictions and healthcare outcomes, may be stored in the User Database 114 for data-driven decision-making and resource allocation.

The Marketing Automation Module 116 is a component of the Healthcare Marketing Automation System 106, designed to streamline and optimize the process of delivering digital media assets and communications to patients and prospective patients using AI algorithms. This module determines which patients and/or prospective patients should receive digital media assets and communications by analyzing patient data and their healthcare journey stage. Based on algorithmic analysis, the module selects appropriate digital media assets and communications to be sent. To ensure the relevance and effectiveness of the digital media assets, the Marketing Automation Module works in conjunction with the Content Generation Module 120 to customize the content as needed using AI techniques. This allows for the creation of tailored messages that address the specific needs and concerns of patients and prospective patients. Once the digital media assets and communications are customized, the Marketing Automation Module schedules the delivery at designated times, ensuring timely and well-coordinated communication. The Messaging Module 118 is then utilized to send digital media assets and messages to the patients and prospective patients through their preferred communication channels.

The Messaging Module 118 is a component of the Healthcare Marketing Automation System 106, responsible for delivering messages to patients and prospective patients through a variety of means using AI algorithms. These means may include, for example, SMS, MMS, voice messages, and video messages. This versatility allows healthcare practitioners to communicate effectively with their patients using the most suitable and preferred channels. To further enhance the messaging experience, the Messaging Module 118 may provide AI-generated customizable scripts that users can modify or adapt to suit their specific needs. These scripts can be used to create tailored, engaging, and informative messages that resonate with patients and prospective patients. In the case of voice and video messaging systems, the customizable scripts can be read aloud by the user, ensuring a personalized touch and human connection in the communications.

The Messaging Module 118 may retrieve the customized content and delivery information from the Marketing Automation Module 116. For example, the Messaging Module 118 may obtain a personalized email newsletter for a patient who has recently been diagnosed with diabetes, or an appointment reminder for a flu vaccination campaign. The Messaging Module 118 may determine the appropriate communication channel for each recipient based on their preferences, available contact information, and insights from the Lead Scoring Module 132. For example, the Messaging Module 118 may choose to send an SMS reminder to a patient with a registered mobile number and a high lead score, or an email to another patient with a registered email address and a lower lead score. The Messaging Module 118 may prepare the content for the selected communication channel, adapting the format if necessary. For example, the Messaging Module 118 may convert a video message into a suitable file format for sending via email or an MMS, or format a chatbot conversation for use within a messaging app. The Messaging Module 118 may send the message to the patient through the chosen communication channel. For example, the Messaging Module 118 may send an appointment reminder via email to a patient who has a scheduled follow-up visit in two weeks, or a promotional message about a new healthcare service through a chatbot in a messaging app. The Messaging Module 118 may record the delivery status, chatbot interactions, and any responses from the recipients, leveraging the Chatbot Module 128 to capture chatbot-related data. For example, the Messaging Module 118 may track whether an email was opened, whether a patient replied to an SMS appointment confirmation request, or the duration and content of a chatbot conversation. The Messaging Module 118 may communicate the delivery and response information, along with any insights from the Correlation Module 130 and Predictive Analytics Module 134, back to the Analytics Module 122 for further analysis. For example, the Messaging Module 118 may provide data on how many patients opened a particular email newsletter, responded to an appointment reminder, or engaged with a chatbot to schedule a follow-up appointment.

The Content Generation Module 120 is a component of the Healthcare Marketing Automation System 106, designed to facilitate the automated creation of customizable marketing and messaging content through the use of generative AI and machine learning tools. This module allows healthcare practitioners to develop a diverse range of content types, which may include, for example, images, voice memos, videos, surveys, articles, scripts, templates, clinical studies, presentations, and documents. These content types can be used individually or in any combination to create engaging and informative materials that resonate with patients and prospective patients. The Content Generation Module 120 also provides AI-generated Content Suggestions, offering ideas and inspiration for content creation based on the user's practice area, patient demographics, and other relevant factors. This feature ensures that the generated content is both relevant and appealing to the target audience. Additionally, the module offers Content Creation Tools, such as templates, design features, and graphics, which enable users to create professional-looking materials with casc.

The Analytics Module 122 is a component of the Healthcare Marketing Automation System 106, designed to measure various aspects of the system in terms of patient and business metrics through analysis using a set of AI algorithms which may, for example, correlate content, messages, and other healthcare journey touchpoints with specific business outcomes, patient outcomes, and other metrics. This module provides valuable insights by evaluating key performance indicators, which may include, for example, engagement (clickthrough, view count, etc.), response rate (replies), conversion (scheduled appointments), retention (predicted vs. actual milestone on healthcare journey), payments, referrals, content types, and campaigns. By analyzing these metrics, the Analytics Module 122 helps healthcare practitioners identify trends, measure the effectiveness of their marketing efforts, and optimize their strategies accordingly using AI-generated recommendations.

Patient Database 124 is a component of the Healthcare Marketing Automation System 106, designed to store and manage patient information. This database includes personally identifying information, such as patient name, birth date, contact information such as phone number, email, etc., and parent or guardian details, to facilitate personalized communications and tailored marketing messages. The Patient Database 124 also stores essential clinical data, including diagnosis, test results, and the patient's healthcare journey stage, providing healthcare practitioners with valuable insights to inform their marketing strategies and patient care plans. This comprehensive repository of patient data helps ensure that healthcare professionals can access and utilize the information they need to deliver targeted and effective marketing campaigns while maintaining a high standard of care for their patients.

The Patient Database 124 may serve as a comprehensive repository for various types of patient data, including but not limited to personal identifying information, clinical data, communication preferences, appointment/attendance data, previously received communications, and engagement data. Furthermore, the Patient Database 124 incorporates data related to the Chatbot Module 128, Correlation Module 130, Lead Scoring Module 132, and Predictive Analytics Module 134 to enhance patient insights and personalize communications. For example, the Patient Database 124 may store personal identifying information such as John Doe's name, date of birth, address, contact number 555-1234, and insurance details with XYZ Insurance Company. It may also store clinical data, including a patient diagnosed with Type 2 diabetes, a recent HbA1c test result of 7.5%, prescribed Metformin, and following a treatment plan that includes regular blood glucose monitoring and dietary changes. The Patient Database 124 retains communication preferences for patients, indicating their preferred channels and frequency of communication, such as a patient who prefers to receive appointment reminders via email rather than phone calls, and educational material about their condition on a monthly basis. Appointment and attendance data, including information about a patient who attended a dermatology appointment on Feb. 1, 2023, and has a follow-up appointment scheduled for Mar. 15, 2023, is also stored in the Patient Database 124. The Patient Database 124 also maintains a record of all marketing and informational materials sent to a patient, along with any personalized content or messages, such as an email sent to a patient on Jan. 15, 2023, containing an article about managing chronic pain, along with a personalized message from their healthcare provider. This database further stores engagement data, such as click-through rates, views, responses, and other interactions with digital media assets and communications, including read receipts for text and email messages. Data from the Chatbot Module 128 can be integrated into the Patient Database 124, allowing healthcare providers to track patient interactions with chatbots, such as frequently asked questions or concerns. For instance, the Patient Database 124 could store a record of John Doe asking the chatbot about managing his Type 2 diabetes symptoms. Similarly, the Correlation Module 130, Lead Scoring Module 132, and Predictive Analytics Module 134 can contribute valuable information to the Patient Database 124. The Correlation Module 130 might identify patterns between patient demographics and preferences for communication channels. The Lead Scoring Module 132 could assign scores to potential new patients based on their likelihood to schedule appointments, and the Predictive Analytics Module 134 may provide predictions about patient behavior or treatment outcomes, such as the likelihood of John Doe adhering to his treatment plan.

The Patient Module 126 is an integral part of the Healthcare Marketing Automation System 106, designed to provide patients with a user-friendly interface for inputting and managing their data. The module may feature, for example, an AI-assisted funnel page that guides patients through the process of entering relevant information, ensuring that essential data is collected and stored in an organized manner. This AI assistance can offer suggestions based on the patient's input, making the data entry process more efficient and accurate. Patients can also view and manage their personal information, such as contact details, to keep their records up-to-date. Once the patient data is entered, the Patient Module 126 securely stores it in the Patient Database 124, making it readily accessible to healthcare practitioners for the purpose of delivering personalized marketing campaigns and tailored patient care.

The Patient Module 126 may enable patients to access the Healthcare Marketing Automation System 106 through a secure, user-friendly interface. For example, a patient with diabetes may log in to the system using their unique credentials, such as an email address and password, on a dedicated patient portal. In another example, a patient with chronic migraines accesses the system through a mobile app. In some embodiments, the Patient Module 126 may allow the patient to input their preferred communication channel (email, text message, phone call, ctc.). In other embodiments, the Patient Module 126 enables the patient to authorize a device to access their data or designate another party, such as a parent, guardian, or spouse, who may be allowed to access their patient data. The Patient Module 126 may present a funnel page or questionnaire, allowing patients to input their personal and clinical data. For example, a new patient with sleep apnea may provide their name, date of birth, contact information, medical history, and insurance details. In another example, a patient with dry eye syndrome may input their preferred communication channel, current medications, and any allergies. The Patient Module 126 may retrieve and display relevant patient data from the Patient Database 124. For example, a patient with diabetes may view their appointment history, treatment plan, and test results after logging into the system. In another example, a patient with chronic migraines can access educational materials, such as articles and videos, tailored to their condition and preferences. The Chatbot Module 128 may also provide personalized recommendations and support based on the patient's data. In some embodiments, the patient data displayed may be based on the users security access level, such that the patient may have one level of access, a guardian or spouse may have a second level of access, and a user or practitioner may have a third level of access. The Patient Module 126 may allow patients to update and manage their personal and clinical data. For example, a patient with sleep apnea may update their contact information, add a new insurance provider, or report a change in their medical condition. In another example, a patient with dry eye syndrome can indicate their preferred appointment times or update their communication preferences. The Lead Scoring Module 132 may use these updates to adjust the patient's lead score, allowing healthcare providers to prioritize their outreach efforts. The Patient Module 126 may securely store any updated patient data in the Patient Database 124. For example, if a patient with diabetes updates their phone number, the system saves the new contact information to the Patient Database 124 for future reference and communication. In another example, if a patient with chronic migraines reports a change in their condition, the Predictive Analytics Module 134 may analyze this new information and provide insights on the patient's treatment progress and potential adjustments to their care plan.

The Chatbot Module 128 is a component of the Healthcare Marketing Automation System 106, designed to facilitate seamless interactions between patients and healthcare practitioners by leveraging AI-powered chatbot technology. The chatbot can be used by both patients and users to access patient and user data, generate content, send, and receive messages, automate marketing campaigns, request clinical information about specific healthcare conditions and healthcare journey stages, and analyze aspects of marketing campaigns via any of the previously described modules. For patients, the Chatbot Module 128 offers an interactive interface to access their personal information, receive personalized healthcare tips, request appointment scheduling, and ask questions about their healthcare journey or specific medical conditions. The AI-driven chatbot can intelligently analyze the patient's query and retrieve relevant information from the system's modules, providing accurate and timely responses. For healthcare practitioners, the Chatbot Module 128 enables efficient management of patient data, streamlining the process of creating and customizing marketing campaigns, and generating content by offering AI-assisted suggestions. The chatbot can also analyze marketing campaign performance metrics by integrating with the Analytics Module 122, allowing practitioners to optimize their strategies accordingly.

The Chatbot Module 128 may initiate interaction with patients or healthcare practitioners when they access the Healthcare Marketing Automation System 106. For example, a patient seeking information about diabetes management may initiate a conversation with the chatbot, or a healthcare practitioner may use the chatbot to access patient records quickly. The Chatbot Module 128 may use natural language processing to understand user queries and provide relevant responses. For example, a patient may ask the chatbot about the side effects of a prescribed medication, and the chatbot responds with accurate information. In another example, a healthcare practitioner may inquire about a patient's last appointment date, and the chatbot provides the necessary details. The Chatbot Module 128 may access and retrieve relevant data from the Patient Database 124 or other system components to provide accurate responses. For example, a patient may inquire about their next appointment, and the chatbot retrieves the appointment information from the Patient Database 124. In another example, a healthcare practitioner may ask for a specific patient's treatment plan, and the chatbot retrieves the information from the appropriate system component. The Chatbot Module 128 may use patient data and artificial intelligence to provide personalized recommendations for patients. For example, a patient may ask the chatbot for suggestions on managing their hypertension, and the chatbot provides tailored advice based on the patient's medical history and lifestyle. In another example, a patient may inquire about exercise options for their chronic back pain, and the chatbot suggests suitable exercises based on the patient's condition and physical abilities. The Chatbot Module 128 may update the Patient Database 124 with any new information obtained during the chatbot interaction. For example, if a patient reports a change in their symptoms or medication, the chatbot stores the updated information in the Patient Database 124. In another example, a healthcare practitioner may provide the chatbot with new clinical data for a patient, such as recent test results, which the chatbot stores in the Patient Database 124 for future reference.

The Chatbot Module 128 may utilize various artificial intelligence (AI) and machine learning (ML) techniques to effectively communicate with patients and healthcare practitioners while maintaining security and accuracy. The Chatbot 128 may incorporate, for example, Natural Language Processing (NLP), a subfield of AI that focuses on understanding and generating human language. The Chatbot Module 128 may use NLP to interpret user queries, extract relevant information, and provide accurate responses. Advanced NLP techniques, such as sentiment analysis, can be employed to understand the context and emotions behind user queries to provide more personalized and empathetic responses. The Chatbot Module 128 may use pre-trained language models, such as GPT-3 or BERT, to generate more accurate and coherent responses. These models can be fine-tuned on domain-specific healthcare data to improve their performance in understanding medical terminology and providing relevant information. The Chatbot Module 128 may employ deep learning techniques, such as recurrent neural networks (RNNs) or transformers, to process and analyze sequential data, like user queries, more effectively. These techniques can help the chatbot understand complex sentences, identify key information, and generate contextually appropriate responses. The Chatbot 128 may incorporate, for example, reinforcement learning, which is an ML approach that trains the chatbot to make decisions by rewarding or penalizing actions based on their outcomes. This technique can be used to optimize the chatbot's responses and provide more relevant information to users over time. Furthermore, to ensure the safety and security of the information provided by the Chatbot Module 128, several measures may be implemented. For example, The Chatbot 128 may incorporate, Healthcare Practitioner Authorization, wherein the Chatbot Module 128 may be programmed to only provide specific instructions or advice from the healthcare practitioner associated with the patient, ensuring that the information given is relevant and accurate. This may include verifying the healthcare practitioner's identity before allowing access to the chatbot or using digital signatures to authenticate the source of the information. The Chatbot 128 may incorporate, for example, Restricting Modifications, wherein the Chatbot Module 128 may be restricted from modifying any information inputted by healthcare practitioners, ensuring the integrity of medical data. Any changes to patient data should be authorized and made directly by the healthcare practitioner or designated staff. The Chatbot 128 may incorporate, for example, Content Filtering, wherein the Chatbot Module 128 can implement content filtering techniques to prevent it from providing erroneous or dangerous information. This may involve using ML algorithms to identify and filter out harmful content or employing a predefined list of prohibited phrases or topics. The Chatbot 128 may incorporate, for example, monitoring and logging, wherein the Chatbot Module 128 may be programmed to monitor and log user interactions to detect any potential issues or security breaches. Regular audits can be conducted to ensure the chatbot is providing accurate information and adhering to security protocols. The Chatbot 128 may incorporate, for example, escalation to human support, wherein the Chatbot Module 128 may include an escalation feature, enabling users to connect with a human support agent or healthcare practitioner in cases where the chatbot cannot provide accurate or appropriate information, or when complex medical advice is required. This ensures that patients receive the necessary assistance and reduces the risk of misinformation. By implementing these AI and ML techniques and security measures, the Chatbot Module 128 can provide accurate, personalized, and secure information to patients and healthcare practitioners while minimizing the risk of errors or security breaches.

The Correlation Module 130 is a component of the Healthcare Marketing Automation System 106, designed to employ AI and ML techniques to establish relationships between business outcomes, patient outcomes, engagement metrics, and various aspects of marketing campaigns automated by the Marketing Automation Module 116, messages created by the Messaging Module 118, content generated by the Content Generation Module 120, analysis generated by the Analytics Module 122, and/or other patient and user data. The module leverages a plurality of and ML correlation approaches to achieve these results, enhancing the effectiveness of marketing campaigns and facilitating data-driven decision-making. Specifically, Correlation Module 130 may employ AI and ML correlation approaches that the Correlation Module 130 may employ include linear regression analysis, decision trees and random forests, neural networks, clustering algorithms, and time series analysis. Linear regression techniques can be used to identify and quantify the strength and direction of the relationships between various campaign aspects and engagement metrics, patient outcomes, or business outcomes. Decision trees and random forests can be utilized to classify and predict the success of different marketing campaign components and strategies based on historical data, enabling healthcare practitioners to optimize their marketing efforts. Neural networks can be leveraged to model complex relationships between marketing campaign variables and outcomes, uncovering hidden patterns and insights that may not be readily apparent using traditional statistical methods. Clustering algorithms can help identify common characteristics and trends by grouping similar patients or marketing campaign elements together, which may impact patient outcomes and business performance. Time series analysis can be utilized to understand and forecast the impact of marketing campaign components on patient outcomes and business performance over time, enabling practitioners to adjust their strategies proactively.

The Lead Scoring Module 132 is a component of the Healthcare Marketing Automation System 106, designed to employ AI and ML techniques to automate lead scoring and nurturing processes for private healthcare practices. This module analyzes data on leads to identify which prospective patients are most likely to convert, helping practices save time and resources while improving the quality of their leads. Specific AI and ML approaches utilized by the Lead Scoring Module 132 include logistic regression, support vector machines (SVM), decision trees, and neural networks. These techniques enable the module to analyze various data points and predict the likelihood of a prospective patient converting based on historical conversion data and other relevant factors. Logistic regression and SVM can classify leads based on the probability of conversion, while decision trees and neural networks can model complex relationships between lead attributes and conversion likelihood. Once the leads are scored, the Lead Scoring Module 132 leverages the Marketing Automation Module 116, Messaging Module 118, and the Content Generation Module 120 for automated nurturing. The Content Generation Module 120 tailors marketing campaigns and messages according to the lead's score, preferences, and position in the sales funnel. This targeted nurturing approach effectively guides leads through the funnel, increasing the likelihood of conversion and ultimately becoming patients. By utilizing these AI and ML approaches, the Lead Scoring Module 132 streamlines the lead scoring and nurturing process for private healthcare practices, resulting in a more efficient and effective patient acquisition process.

The Lead Scoring Module 132 may initialize the process of assigning scores to patients based on their engagement data and potential value to the healthcare practice. The lead score may be assigned to a specific patient or a group of patients sharing a similar characteristics, such as the patient demographics, medical history, interest level with a certain type of medical topic, the propensity for a type of communication channel or engaging with a type of digital media asset. For example, the module gathers data from the Patient Database 124 and other modules, such as the Chatbot Module 128 and Predictive Analytics Module 134, to evaluate the patients' interactions with digital media assets and their likelihood of scheduling appointments or undergoing treatments. In one example, a patient named Jane has visited the dermatology practice's website multiple times, clicked on several articles about acne treatment, and interacted with the Chatbot Module 128 to ask questions about available treatment options. Based on these engagement statistics, the Lead Scoring Module 132 assigns Jane a high lead score, indicating her strong interest in the practice's services. In another example, another patient, Tom, has received emails from a dental clinic about teeth whitening procedures but has not opened any of the messages. The Lead Scoring Module 132 assigns Tom a low lead score due to his lack of engagement with the digital media assets.

The Lead Scoring Module 132 may apply a lead scoring algorithm, which may use AI/ML techniques, to analyze patients' engagement data and calculate their lead scores. The algorithm takes into account various factors, such as demographics, medical history, engagement with digital media assets, and interactions with the chatbot. In one example, Jane's high engagement with the dermatology practice's digital media assets and interactions with the Chatbot Module 128 are used by the lead scoring algorithm to calculate her lead score. The algorithm considers her age, medical history, and the frequency of her interactions with the content, resulting in a high lead score. In another example, Tom's lack of engagement with the dental clinic's emails and the absence of interactions with the Chatbot Module 128 are used by the lead scoring algorithm to calculate his lead score. The algorithm considers his age, medical history, and lack of interest in engaging with the digital media assets, resulting in a low lead score.

The Lead Scoring Module 132 may integrate the calculated lead scores with other modules of the Healthcare Marketing Automation System 106 to optimize marketing strategies and improve patient outcomes. In one example, The Analytics Module 122 uses Jane's high lead score to suggest that the dermatology practice should target her with personalized content about acne treatments, leading to a higher likelihood of Jane scheduling an appointment and receiving the appropriate treatment. The Predictive Analytics Module 134 may identify a pattern of low lead scores for patients like Tom, who received teeth whitening emails. The module recommends the dental clinic to adjust its marketing strategy and test alternative content formats or messaging to improve engagement and increase the likelihood of these patients scheduling appointments.

The Lead Scoring Module 132 may evaluate and update the lead scores based on new patient engagement data or changes in patients' circumstances. This allows the system to adapt and refine its marketing strategies over time. In one example, after receiving personalized content about acne treatments, Jane schedules an appointment with the dermatology practice. The Lead Scoring Module 132 updates her lead score to reflect this positive outcome and informs the Analytics Module 122 to monitor her engagement with future digital media assets. In another example, Following the dental clinic's adjustments to its marketing strategy, Tom begins engaging with the new content format and schedules a teeth whitening appointment. The Lead Scoring Module 132 updates Tom's lead score to reflect his increased engagement, allowing the system to tailor future marketing efforts accordingly. In one embodiment, Jane, a 25-year-old woman, demonstrates interest in a dermatology clinic's acne treatment options. The Lead Scoring Module 132 collects and analyzes her engagement data, including multiple website visits, email opens, chatbot interactions, and social media engagement. For example, Lead Scoring Module 132 collects and analyzes the following engagement data to calculate her lead score; Website visits: 10 visits in the past month; Email opens: Opened 5 out of 6 acne-related emails; Chatbot interactions: 3 sessions, inquiring about treatment options; Social media engagement: Liked and shared 2 acne treatment posts. To calculate Jane's lead score, the module employs a weighted scoring system based on the relative importance of each engagement factor. The points assigned to each factor are combined to produce a total lead score. For example, Website visits: 10 points per visit (100 points); Email opens: 20 points per email opened (100 points); Chatbot interactions: 30 points per session (90 points); Social media engagement: 10 points per engagement (20 points). Jane's total lead score may be calculated thusly: 100+100+90+20=310 points. In this example, Jane's high lead score indicates a strong interest in the clinic's offerings. Consequently, the Healthcare Marketing Automation System 106 utilizes her lead score to optimize outcomes for both the business and Jane by sending her personalized content related to acne treatments, discounts on consultations, and reminders about scheduling appointments. As a result, Jane schedules an appointment with the clinic, receives appropriate treatment, and becomes a satisfied, long-term patient. The dermatology clinic benefits from increased revenue and a loyal customer who may refer friends and family. In another embodiment, Tom, a 45-year-old man, receives emails from a dental clinic about teeth whitening procedures. The Lead Scoring Module 132 collects and analyzes his engagement data, including a single website visit, one email open, and no chatbot interactions or social media engagement. For example, Tom's engagement data is as follows; Website visits: 1 visit in the past month; Email opens: Opened 1 out of 5 teeth whitening emails; Chatbot interactions: None; Social media engagement: None. The module utilizes the same weighted scoring system as in the first example to calculate Tom's lead score. For example, Website visits: 10 points per visit (10 points); Email opens: 20 points per email opened (20 points); Chatbot interactions: 0 points; Social media engagement: 0 points. Tom's total lead score can be calculated thusly: 10+20+0+0=30 points. In this instance, Tom's low lead score indicates the need to adjust the marketing strategy to better engage him. The Healthcare Marketing Automation System 106 suggests testing alternative content formats, such as video testimonials or informational infographics about teeth whitening procedures. After implementing these changes, Tom engages with the new content and schedules an appointment for teeth whitening. The dental clinic benefits from increased revenue, while Tom receives a service that improves his dental health and appearance.

The Predictive Analytics Module 134 is a component of the Healthcare Marketing Automation System 106, designed to employ AI and ML techniques to help private healthcare practices anticipate patient behavior and trends before they occur. By analyzing data on past patient behaviors, the module can make predictions about future behaviors, enabling practices to adjust their strategies accordingly. Specific AI and ML approaches utilized by the Predictive Analytics Module 134 include time series analysis, linear regression, decision trees, neural networks, and clustering algorithms. These techniques enable the module to analyze historical data, identify patterns and trends, and project patient behaviors and preferences in the future. Time series analysis and linear regression can model temporal trends in patient data, while decision trees, neural networks, and clustering algorithms can capture complex relationships between various patient attributes and behaviors. For instance, private healthcare practices can leverage the insights generated by the Predictive Analytics Module 134 to determine the best time to launch a new product or service, based on historical data and projected patient trends. This data-driven approach allows practices to optimize their marketing strategies, product offerings, and services to better meet the needs of their patients, resulting in improved patient satisfaction and business outcomes. By utilizing these AI and ML approaches, the Predictive Analytics Module 134 empowers private healthcare practices to proactively address patient needs and trends, enabling them to stay ahead of the competition and provide exceptional patient care.

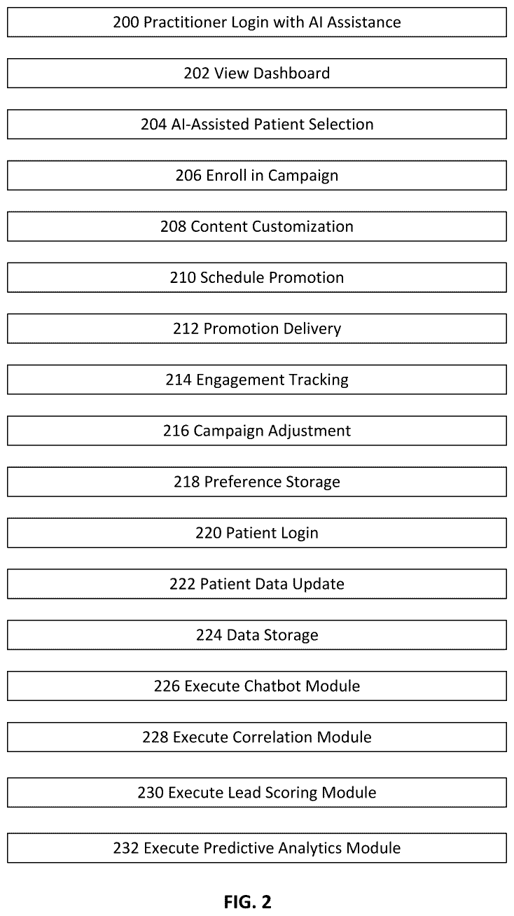

FIG. 2 displays base module 108. The process begins at step 200, the practitioner logs in to the Healthcare Marketing Automation System 106 using their unique credentials, granting them access to the system's features and patient data. An AI-driven authentication process can enhance security by analyzing user behavior and login patterns. For example, Dr. Smith logs in to schedule a flu vaccination campaign for eligible patients in their practice. At step 202, upon logging in, the practitioner is presented with the AI-enhanced Dashboard, displaying an overview of patient data, marketing campaigns, messages, content, analytics, and other relevant information. The dashboard may use AI to prioritize and present information based on individual practitioner preferences and usage patterns. For example, Dr. Smith sees that the most recent diabetes management campaign has a high engagement rate and several scheduled appointments. At step 204, the practitioner selects a specific patient from the patient list or by searching Patient Database 124 to view, update, or manage their data and enroll them in a marketing campaign. AI algorithms can help identify patients who may benefit from specific campaigns based on their healthcare journey or other factors. For example, Dr. Smith selects a patient from the list, such as John Doe, to review their healthcare journey and determine if they are eligible for any new campaigns, like the flu vaccination campaign. At step 206, the practitioner enrolls the selected patient in a marketing campaign by choosing from existing AI-optimized campaigns or creating a new one tailored to the patient's healthcare journey stage. AI can help identify the most effective campaign strategies based on patient data and historical outcomes. For example, Dr. Smith enrolls John Doe in the flu vaccination campaign, as he is eligible and has not yet received the vaccine. At step 208, the practitioner customizes the content of the marketing messages for the selected patient, utilizing the AI-powered Content Generation Module 120 to create engaging and informative materials. AI algorithms can generate personalized content based on the patient's preferences, health conditions, and other factors. Dr. Smith customizes the content of the flu vaccination reminder message, adding a personal touch by mentioning John Doe's recent visit and the importance of the vaccine for his specific health conditions. At step 210, the practitioner schedules the delivery of the customized marketing message at a designated date and time, ensuring timely and relevant communication with the patient. AI algorithms can determine the optimal time to send messages based on patient engagement patterns and other factors. For example, Dr. Smith schedules the flu vaccination reminder message to be sent to John Doe on the following Monday, as it is the optimal time to maximize engagement. At step 212, the Messaging Module 118 sends the scheduled marketing message to the patient via their preferred communication channel, such as email, SMS, or voice message. AI algorithms can analyze patient preferences and engagement patterns to optimize message delivery. For example, the Messaging Module 118 sends the customized flu vaccination reminder to John Doe via SMS, as indicated in his preferred communication channel. At step 214, the Analytics Module 122 tracks engagement metrics, such as clickthrough rates, view counts, responses, and conversions, to assess the effectiveness of the marketing campaign. AI algorithms can analyze engagement data in real-time, providing deeper insights and predictive analytics. For example, Dr. Smith monitors the engagement metrics of the flu vaccination campaign, noticing that many patients have clicked on the provided link and scheduled their vaccinations. At step 216, based on the AI-generated analytics data, the practitioner adjusts the marketing campaign, tailoring the content, delivery, or other factors to optimize results and improve patient engagement. AI algorithms can provide data-driven recommendations for campaign adjustments based on patient engagement patterns and other factors. For example, based on the tracked engagement, Dr. Smith adjusts the campaign by changing the message content to emphasize the limited availability of flu vaccine appointments, motivating patients to schedule more promptly. At step 218, the User Database 114 stores the practitioner's preferences, settings, and other relevant information, ensuring a personalized and efficient user experience. AI algorithms can analyze practitioner usage patterns to optimize system settings and tailor the user experience accordingly. For example, the User Database 114 stores Dr. Smith's preferences, such as message templates and preferred communication channels, for future use in the Healthcare Marketing Automation System 106.

At step 220, the patient logs in to the Healthcare Marketing Automation System 106 through their patient device, accessing their patient data and managing their healthcare information. AI-driven authentication processes can provide additional security and enhance the user experience. For example, John Doe logs in to the patient portal of the Healthcare Marketing Automation System 106 to review the flu vaccination reminder message and access his healthcare information. At step 222, the patient updates their personal and clinical data, such as contact information, medical history, or appointment details, ensuring accurate and up-to-date information. AI algorithms can validate and check the consistency of the updated data, flagging potential errors or discrepancies. For example, John Doe updates his contact information, including his phone number and email address, to ensure he receives future communications from Dr. Smith's practice. At step 224, the updated patient data is stored in Patient Database 124, allowing healthcare practitioners to access and manage the most recent information for better patient care and communication. AI algorithms can optimize data storage and retrieval, ensuring fast and efficient access to patient data. For example, the Healthcare Marketing Automation System 106 stores John Doe's updated contact information in the Patient Database 124, ensuring accurate communication for future campaigns and messages. At step 226, the Chatbot Module 128 is executed, allowing patients and users to interact with the Healthcare Marketing Automation System 106 in a conversational manner. For example, John Doe can use the chatbot to access his patient data, generate content, send and receive messages, automate marketing campaigns, request clinical information, and analyze aspects of marketing campaigns via other modules. At step 228, the Correlation Module 130 is executed, employing AI and ML techniques to correlate business outcomes, patient outcomes, and engagement metrics with aspects of marketing campaigns, messages, content, analytics, and other patient and user data. For example, the Correlation Module 130 may analyze data from the Marketing Automation Module 116 and Messaging Module 118 to identify which marketing strategies are most effective at improving patient outcomes and generating referrals. At step 230, the Lead Scoring Module 132 is executed, utilizing AI and ML techniques to analyze data on leads and identify which prospects are most likely to become patients. This module can also automate nurturing using the Marketing Automation Module 116, Messaging Module 118, Content Generation Module 120, and other modules to guide leads through the sales funnel. For example, the Lead Scoring Module 132 might prioritize prospects with certain health conditions and engagement patterns, enabling Dr. Smith to focus on the most promising leads. At step 232, the Predictive Analytics Module 134 is executed, using AI and ML techniques to anticipate patient behavior and trends by analyzing data on past patient behaviors. For example, Dr. Smith can use the insights generated by the Predictive Analytics Module 134 to determine the best time to launch a new service, such as a diabetes management program, based on historical data and projected patient needs.

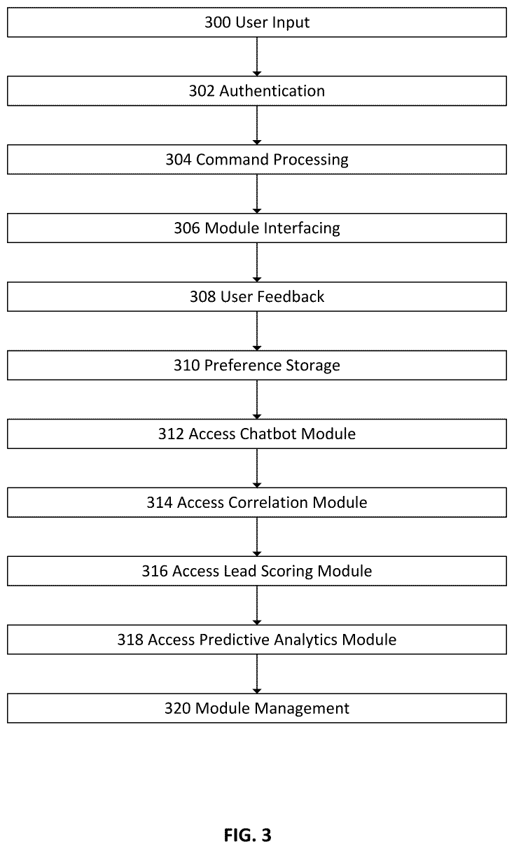

FIG. 3 illustrates a user module 110, according to an embodiment. The process begins at step 300, the User Module 110 receives user input, such as login credentials, commands, or data from a healthcare practitioner, while AI algorithms can provide autocomplete suggestions and error-checking for enhanced user experience. For example, Dr. Smith enters their login credentials, selects a specific patient from the patient list, and modifies a marketing message template for a flu vaccination campaign with AI-generated suggestions. At step 302, the User Module 110 uses AI algorithms to authenticate the practitioner's credentials more securely, incorporating techniques like biometric authentication, anomaly detection, or behavioral analysis to verify their identity and grant access to the system. Dr. Smith's unique username, password, and other authentication factors are verified, ensuring authorized access to the system and patient information. At step 304, the User Module 110 processes user commands with the help of AI algorithms, potentially offering suggestions for optimizing marketing campaigns or identifying patient trends based on historical data. For example, Dr. Smith selects John Doe, enrolls him in a flu vaccination campaign, customizes the reminder message, and schedules it to be sent on a specific date and time, with AI-generated recommendations for content and timing. At step 306, the User Module 110 interfaces with other modules, such as the Content Generation Module 120 or Analytics Module 122, more efficiently using AI algorithms to execute requested actions based on user commands. Upon receiving a user command from Dr. Smith to enroll John Doe in a flu vaccination campaign, the User Module 110 communicates with the Marketing Automation Module 116 to create a tailored marketing message with AI-generated content and schedule its delivery via the Messaging Module 118. At step 308, the User Module 110 uses AI algorithms to provide more insightful and relevant user feedback, such as the results of a command, error messages, or data-driven recommendations for campaign improvements. Dr. Smith receives confirmation that John Doe has been enrolled in the flu vaccination campaign and can view AI-driven engagement metric predictions and improvement suggestions for the campaign. At step 310, the User Module 110 stores user preferences and settings, such as their preferred dashboard layout or notification preferences, in the User Database 114, using AI algorithms to learn from the practitioner's behavior and further tailor the system to their needs. Dr. Smith's preferred communication channels, message templates, and frequently used marketing campaign settings are saved and analyzed in User Database 114, streamlining the process for future campaigns and system use. At step 312, the User Module 110 allows the healthcare practitioner to access the Chatbot Module 128, which provides an interactive AI-driven chatbot for patient engagement and support. For example, Dr. Smith can access the Chatbot Module 128 to review and manage the chatbot's responses and settings to optimize patient interactions. At step 314, the User Module 110 enables the practitioner to access the Correlation Module 130, which employs AI algorithms to identify correlations and patterns in patient data, campaign performance, and other relevant metrics. For example, Dr. Smith can access the Correlation Module 130 to discover trends in patient behavior and preferences, enabling more effective marketing campaigns and communication strategies. At step 316, the User Module 110 allows the healthcare practitioner to access the Lead Scoring Module 132, which utilizes AI algorithms to assign scores to patients based on their likelihood to engage with marketing campaigns, schedule appointments, or adhere to treatment plans. For example, Dr. Smith can access the Lead Scoring Module 132 to prioritize patients for targeted marketing campaigns or follow-up communications. At step 318, the User Module 110 provides the practitioner access to the Predictive Analytics Module 134, which leverages AI algorithms to predict patient behavior, campaign performance, and healthcare outcomes based on historical data and trends. For example, Dr. Smith can access the Predictive Analytics Module 134 to forecast the effectiveness of a flu vaccination campaign and make data-driven decisions on campaign adjustments and resource allocation. At step 320, the User Module 110 facilitates seamless integration and management of the Chatbot Module 128, Correlation Module 130, Lead Scoring Module 132, and Predictive Analytics Module 134, allowing the practitioner to access and utilize these AI-driven modules to optimize patient engagement and communication. For example, Dr. Smith can use the User Module 110 to switch between the different AI-driven modules, applying insights and recommendations to enhance marketing campaigns and overall patient care.

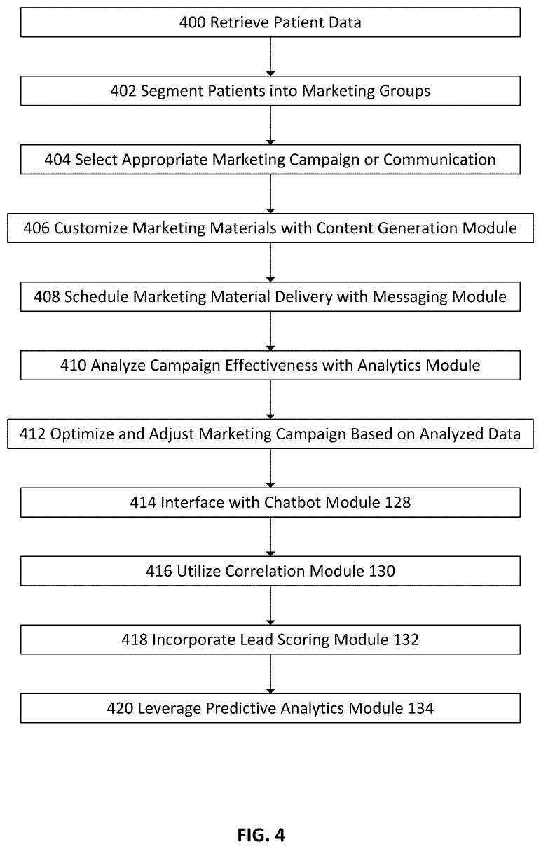

FIG. 4 illustrates a marketing automation module 116, according to an embodiment. The process begins at step 400, the Marketing Automation Module 116 retrieves patient data from the Patient Database 124, such as a patient's diagnosis, healthcare journey stage, and previous engagement history. For example, the module may identify patients who have recently been diagnosed with hypertension or those who have a scheduled flu vaccination appointment. In an exemplary embodiment of a healthcare journey for a myopia patient, the patient may initially experience difficulties with their vision, particularly in discerning distant objects. This journey could encompass several stages, as previously mentioned.

At step 402, the module segments patients into appropriate marketing groups based on their healthcare journey stage and other relevant factors. For example, patients with hypertension may be segmented into groups such as newly diagnosed, ongoing management, and high risk. Meanwhile, patients who are due for a flu vaccination can be segmented based on factors such as age, previous vaccination history, and current health conditions. At step 404, the Marketing Automation Module 116 selects an appropriate marketing campaign or communication for each patient group. For example, newly diagnosed patients may receive a campaign focusing on education and lifestyle changes, while high-risk patients may receive more targeted information on medication management and regular check-ups. The Chatbot Module 128 may also be integrated into the campaign, providing patients with interactive guidance and support. At step 406, the module customizes the selected digital media assets or communications based on individual patient data using the Content Generation Module 120. For example, the module may personalize a campaign for a high-risk hypertension patient by including their name, recent test results, and a reminder to schedule their next appointment. The Correlation Module 130 might identify correlations between patient conditions and treatment outcomes to further tailor the content. At step 408, the Marketing Automation Module 116 schedules the delivery of the digital media assets or communications to the patients using the Messaging Module 118. For example, the module may send an educational email to newly diagnosed patients a week after their initial diagnosis, followed by a series of targeted messages over the next few months. The Lead Scoring Module 132 could prioritize patients based on their level of engagement or likelihood to convert, allowing for more focused marketing efforts.

At step 410, the module analyzes the effectiveness of the marketing campaign or communication using the Analytics Module 122, tracking metrics such as engagement, response rate, and conversion. For example, the module may determine that the educational email for newly diagnosed patients has a high open rate and leads to an increased number of scheduled appointments. Predictive Analytics Module 134 may provide insights into future trends, such as potential shifts in patient behavior or preferences. At step 412, the Marketing Automation Module 116 adjusts the marketing campaign or communication based on the analyzed data to optimize its effectiveness. For example, if the module determines that patients are more likely to open an email in the evening, it may adjust the sending time to increase engagement. Additionally, AI-driven recommendations may be provided to help healthcare practitioners refine their marketing strategies based on ongoing analysis and insights from the Chatbot Module 128, Correlation Module 130, Lead Scoring Module 132, and Predictive Analytics Module 134.

At step 414, the Marketing Automation Module 116 interfaces with the Chatbot Module 128 to incorporate chatbot interactions into marketing campaigns. For example, the module may deploy a chatbot for a flu vaccination campaign to answer frequently asked questions, schedule appointments, or provide personalized recommendations based on patient input. At step 416, the Marketing Automation Module 116 utilizes the Correlation Module 130 to identify patterns and relationships between various patient factors and their responses to marketing campaigns. For example, the module may identify that patients with specific health conditions are more likely to engage with a particular type of content, which can then be used to optimize future campaigns for those patients.

At step 418, the Marketing Automation Module 116 incorporates the Lead Scoring Module 132 to assign scores to patients based on their likelihood to engage with marketing campaigns, schedule appointments, or participate in a specific healthcare program. For example, the module may prioritize high-scoring patients for targeted campaigns, ensuring efficient allocation of marketing resources. At step 420, the Marketing Automation Module 116 leverages the Predictive Analytics Module 134 to forecast patient behavior, marketing campaign performance, and overall patient engagement. For example, the module may use predictive analytics to identify optimal times to send digital media assets, anticipate patient attrition, or estimate the potential return on investment for a given marketing campaign.

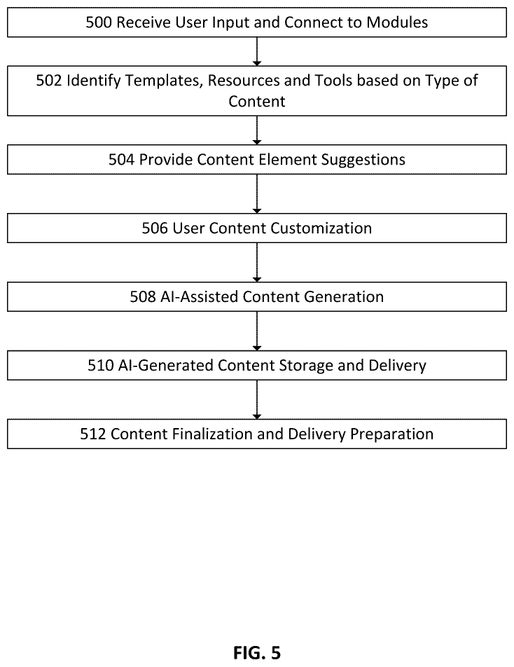

FIG. 5 illustrates a content generation module 120, according to an embodiment. The process begins at step 500, the Content Generation Module 120 receives input from the user, such as a desired type of content or a specific marketing message to be generated. For example, an orthopedic surgeon may request an interactive quiz on proper posture and ergonomics for their patients with back pain, or a pediatrician may ask for an engaging infographic on the importance of vaccinations for children. The Content Generation Module 120 integrates artificial intelligence (AI) and machine learning (ML) algorithms to analyze patient data, practitioner data, and engagement data, which helps in generating more relevant and personalized content for the target audience. Additionally, the module connects with the Chatbot Module 128, Correlation Module 130, Lead Scoring Module 132, and Predictive Analytics Module 134 to gather insights, analyze user interactions, and score leads, further optimizing the content based on audience preferences and behavior. For instance, the Content Generation Module 120 may use AI to analyze the responses to the interactive quiz on posture and ergonomics, identifying common misconceptions and knowledge gaps among the patients. This information can then be used to create more targeted and relevant content, such as blog posts or videos addressing those specific concerns. In another embodiment, the module can analyze the engagement data for the infographic on vaccinations, determining the most effective visual elements and messaging that resonate with the target audience. This information can be used to further optimize future content on vaccinations or other related topics. The Chatbot Module 128 can also be leveraged to interact with users in real-time, gathering additional insights and feedback on the generated content, which can then be used to enhance future content creation.

At step 502, the Content Generation Module 120 utilizes AI and machine learning algorithms to identify available content templates, resources, and tools based on the user input related to the content type. For instance, when a healthcare practitioner requests an educational video on dental hygiene for a specific patient group, the module may provide a list of video templates suitable for creating this video. In addition, the Content Generation Module 120 can connect with the Chatbot Module 128, Correlation Module 130, Lead Scoring Module 132, and Predictive Analytics Module 134 to enhance the content creation process. For example, the module could use insights from the Correlation Module 130 to identify trending topics among the patient group and incorporate them into the video. In another example, the Content Generation Module 120 may work in conjunction with the Lead Scoring Module 132 to prioritize content creation based on patient engagement and interest levels. For instance, if a chiropractic clinic is generating content for two separate patient groups-those interested in spinal decompression therapy and those interested in sports injury rehabilitation—the module could prioritize content creation for the group with higher engagement scores. In other aspects, the Content Generation Module 120 can use AI and machine learning to identify created content that matches the user request and customize it using patient data and/or practitioner data. For example, an instructional video for applying myopia treatments, such as Ortho-K, may be customized with a title screen containing the practitioner's name and practice logo. The text and audio overlay for the video could be selected based on the patient's age, e.g., one set of audios for 5 to 10-year-olds, another set for 11 to 16 years, and a third set for 17 and over, ensuring the content is age-appropriate for the audience. By connecting with the Chatbot Module 128, the module can also collect user preferences and feedback to refine content suggestions, providing a more tailored and engaging experience for both patients and healthcare practitioners.

At step 504, the Content Generation Module 120 leverages AI and machine learning algorithms to provide content element suggestions and customization options based on the user's preferences, patient data, and the identified content templates, resources, and tools. For example, the module might offer various video styles, background music choices, and on-screen text templates for a dental hygiene video. By connecting with the Chatbot Module 128, the module can gather user preferences and feedback to further refine content suggestions, ensuring a more personalized and engaging experience for both patients and healthcare practitioners. For instance, the module may analyze previous interactions between the user and the chatbot to recommend content that has resonated well with similar patients in the past. The Content Generation Module 120 can also collaborate with the Correlation Module 130, Lead Scoring Module 132, and Predictive Analytics Module 134 to enhance content suggestions. For instance, by using insights from the Predictive Analytics Module 134, the module could identify patients more likely to engage with specific content types and recommend content accordingly. In one example, the Content Generation Module 120 may suggest content for a parent of a child with myopia who has not yet scheduled a treatment consultation. The module could emphasize the importance of treating myopia early after diagnosis to prevent the progression of the disorder and additional symptoms. This content suggestion might be based on data from the Lead Scoring Module 132, which identifies patients who are more likely to schedule a consultation after receiving this type of content. In another example, the Content Generation Module 120 might provide content suggestions for a physiotherapy clinic, offering various exercise demonstration video styles and voiceover options for different age groups or conditions. The module could analyze patient engagement data from the Correlation Module 130 to identify which video styles and voiceover options have been most effective in the past, enabling the clinic to make informed decisions when customizing their content.

At step 506, the user customizes the content as needed using the provided suggestions and options, which have been generated based on AI and machine learning algorithms. The module may collaborate with the Chatbot Module 128, Correlation Module 130, Lead Scoring Module 132, and Predictive Analytics Module 134 to create a more seamless and efficient content customization experience for the user. For example, a healthcare practitioner may choose a specific video style, background music, and on-screen text for a dental hygiene video aimed at children. By incorporating data from the Correlation Module 130, the Content Generation Module 120 might recommend video styles that have a higher engagement rate among similar target audiences. In another example, the user could customize a video on managing diabetes to include an excerpt and a reference to a recent clinical study that supports a particular treatment approach. The Content Generation Module 120 could utilize AI algorithms to analyze relevant studies and identify the most impactful excerpts for the target audience. This customization process may also involve the Predictive Analytics Module 134, which could help predict which studies and excerpts are likely to resonate with specific patient segments. In some aspects, the Chatbot Module 128 may play a role in streamlining the customization process. For instance, the healthcare practitioner could interact with the chatbot to quickly adjust content elements such as video style or background music, without having to navigate through complex menus or interfaces. The chatbot could also provide real-time feedback and suggestions based on user preferences and past engagement data from the Lead Scoring Module 132, further enhancing the content customization experience for the user.

At step 508, the Content Generation Module 120 generates customized content based on the user's selections, leveraging artificial intelligence and machine learning algorithms to optimize the final output. The module may collaborate with the Chatbot Module 128, Correlation Module 130, Lead Scoring Module 132, and Predictive Analytics Module 134 to ensure the content is tailored to maximize engagement and effectiveness. For example, the module may create a final dental hygiene video for children, incorporating the chosen video style, background music, and on-screen text. The Content Generation Module 120 could use AI algorithms to analyze past engagement data from the Lead Scoring Module 132 to optimize the video's pacing and structure, ensuring it captures and retains the target audience's attention. In some aspects, the Content Generation Module 120 may generate several different versions of a piece of content, e.g., the same dental hygiene video for children using a plurality of video styles, a plurality of background music tracks, and variations of on screen text, and allow the Analytics Module 122 and/or Correlation Module 130 determine which video elements created the highest engagement metrics for all or some types of patients, thereby providing the Predictive Analytics Module 134 with additional data that allows it to create stronger predictions of parameters of future pieces of content that may be more successful. In another example, the Content Generation Module 120 could generate a series of personalized email campaigns for a physical therapy clinic, highlighting specific exercises and recovery tips for patients with varying conditions. The module might utilize the Predictive Analytics Module 134 to determine the best time to send each email, maximizing the chances of recipients opening and engaging with the content. In some aspects, the Content Generation Module 120 may generate variations of the email campaigns and allow the Analytics Module 122 and/or Correlation Module 130 determine which email campaigns are more successful. The Chatbot Module 128 may assist the user during the content generation process by providing real-time feedback on their selections, answering questions, or offering suggestions based on the Correlation Module 130's analysis of previous successful content. This collaboration ensures that the content generated is not only tailored to the user's preferences but also optimized for the target audience's engagement and satisfactions.

At step 510, the Content Generation Module 120 saves and stores the generated content for later use or immediate delivery through the Messaging Module 118. The module leverages artificial intelligence and machine learning algorithms to offer content customization options and suggestions for various private healthcare practices, incorporating connections with the Chatbot Module 128, Correlation Module 130, Lead Scoring Module 132, and Predictive Analytics Module 134. For example, a dental hygiene video may be saved in a specific marketing campaign folder and made available for delivery to the targeted patient group. The Content Generation Module 120 generates content that is highly relevant and tailored to each patient's needs by accessing patient data, such as age, condition, and treatment history. In another example, such as a myopia clinic, the Content Generation Module 120 can create personalized educational content, like videos or articles, discussing myopia prevention and management. By considering patient engagement data and communication preferences, the module ensures the content is delivered effectively and engages the patient. In another example, such as a dry eyes clinic, the module generates personalized articles and images discussing the causes of dry eyes, preventive measures, and available treatment options. The content is created based on patient data and communication preferences, ensuring it is relatable, engaging, and delivered through the preferred channels. In another example, a dermatology clinic can utilize the Content Generation Module 120 to create customized videos and presentations addressing various skin conditions and treatments. By accessing patient data and considering engagement data, the module tailors the content's length, format, and style to maximize patient engagement. In another example, such as a physical therapy center, the module creates voice memos, videos, and text documents with exercise instructions and recommendations, considering patient data, communication preferences, and engagement data to ensure effective and personalized content delivery. In another example, a nursing home can leverage the Content Generation Module 120 to generate personalized newsletters, surveys, and clinical studies for residents and their families, using patient data to make the content informative and engaging. In another example, such as a mental health and addiction treatment center, the Content Generation Module 120 generates content like articles, scripts, and presentations on various mental health conditions and addiction recovery strategies. The module utilizes patient data, diagnosis, treatment history, and engagement data to generate highly relevant and personalized content, considering communication preferences for effective delivery.

At step 512, the Content Generation Module 120 completes the personalized content creation process from previous steps and gets it ready for delivery. The content can either be sent directly to the Messaging Module 118 for immediate distribution or stored within the Healthcare Marketing Automation System 106 for future use. For example, a myopia doctor may schedule a series of informative articles on myopia prevention and management to be sent to their patients at specific intervals. The Content Generation Module 120 saves these articles and delivers them to the Messaging Module 118 at the designated times. In the case of a dental clinic, the Content Generation Module 120 may finalize an educational video about dental hygiene for a specific patient group. The module saves the video and sends it to the Messaging Module 118 for immediate distribution or schedules it for later delivery based on the clinic's marketing strategy. For a physical therapy center, the module may finalize a set of personalized exercise instructions and recommendations for patients recovering from specific injuries. The Content Generation Module 120 prepares the content for delivery, either immediately or at designated times, to ensure patients receive timely and relevant information throughout their recovery process.

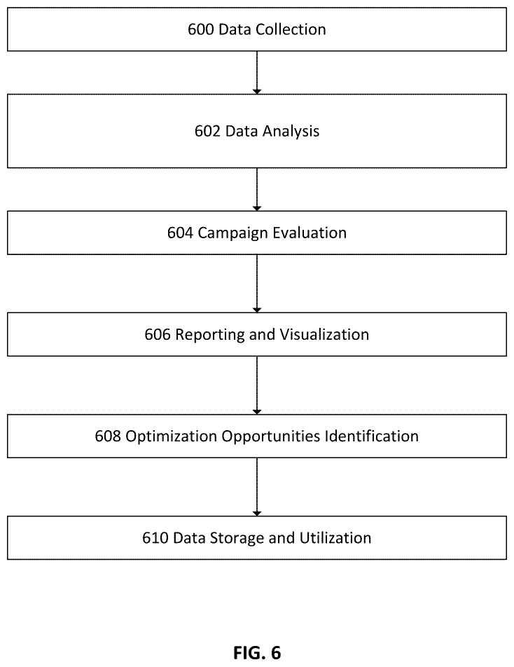

FIG. 6 illustrates an analytics module 122, according to an embodiment. The process begins at step 600. The Analytics Module 122 collects data concerning patient interactions, marketing campaigns, and messaging within the Healthcare Marketing Automation System 106. The module employs artificial intelligence and machine learning algorithms to analyze the collected data while incorporating connections with the Chatbot Module 128, Correlation Module 130, Lead Scoring Module 132, and Predictive Analytics Module 134 to provide comprehensive insights. For example, the module gathers information on message open rates, click-through rates, and patient responses for a myopia doctor's email campaign. For a dental clinic running a social media campaign, the Analytics Module 122 collects data related to user engagement, such as likes, shares, comments, and link clicks. The module also analyzes the effectiveness of the content and identifies trends to optimize future marketing efforts. The connections with the Chatbot Module 128, Correlation Module 130, Lead Scoring Module 132, and Predictive Analytics Module 134 allow for a better understanding of user behavior and preferences, helping the dental clinic refine their targeting and messaging strategies. In the case of a physical therapy center, the Analytics Module 122 gathers data on patient interactions with personalized exercise instructions and recommendations sent through the Messaging Module 118. The module collects information on content views, completion rates, and feedback from patients, helping the center evaluate the effectiveness of their communication and adjust their strategies accordingly. The integration with the Chatbot Module 128, Correlation Module 130, Lead Scoring Module 132, and Predictive Analytics Module 134 ensures a comprehensive analysis of the data, allowing for improved patient engagement and satisfaction.

At step 602, the Analytics Module 122 processes and analyzes the collected data using artificial intelligence and machine learning algorithms to generate valuable insights and metrics. By incorporating connections with the Chatbot Module 128, Correlation Module 130, Lead Scoring Module 132, and Predictive Analytics Module 134, the module enhances the overall analysis, providing actionable insights for healthcare providers to optimize their marketing and communication efforts. For example, it calculates the average response rate for a myopia doctor's email campaign and identifies which types of content have the highest engagement. In the case of a dermatology clinic's marketing campaign, the Analytics Module 122 analyzes data related to patient interactions with various types of content, such as articles, infographics, and videos. The module calculates metrics like engagement rates, conversion rates, and dwell time for each content type. By connecting with the Chatbot Module 128, Correlation Module 130, Lead Scoring Module 132, and Predictive Analytics Module 134, the Analytics Module can provide insights on content preferences and how they relate to patient demographics, allowing the dermatology clinic to tailor their future marketing efforts for better engagement and conversion. For a mental health and addiction treatment center, the Analytics Module 122 processes data from a series of webinars and online workshops provided to patients. The module calculates metrics like attendance rate, drop-off rate, and participant feedback. With the integration of the Chatbot Module 128, Correlation Module 130, Lead Scoring Module 132, and Predictive Analytics Module 134, the Analytics Module can identify trends and correlations between patient demographics, their level of engagement, and the effectiveness of the webinars or workshops. This information helps the treatment center refine their educational offerings and improve patient outcomes.

At step 604, the Analytics Module 122 utilizes artificial intelligence and machine learning algorithms to compare the generated metrics to predefined goals or benchmarks, evaluating the effectiveness of marketing campaigns and messages. By incorporating connections with the Chatbot Module 128, Correlation Module 130, Lead Scoring Module 132, and Predictive Analytics Module 134, the module can provide a comprehensive assessment of campaigns, offering insights into areas for improvement or optimization. In the case of a dental clinic's social media marketing campaign, the Analytics Module 122 compares the engagement rates, reach, and conversions to industry benchmarks or the clinic's previous campaigns. By connecting with the Chatbot Module 128, Correlation Module 130, Lead Scoring Module 132, and Predictive Analytics Module 134, the Analytics Module can identify trends and correlations between the campaign's effectiveness and factors that relate to an element of the content, delivery of the content, and a type of patient, such as content type, posting times, and audience demographics. This information enables the dental clinic to refine their marketing strategy for better results. For a physical therapy center's email marketing campaign, the Analytics Module 122 evaluates the campaign's effectiveness by comparing open rates, click-through rates, and conversion rates to predefined goals or industry averages. The integration with the Chatbot Module 128, Correlation Module 130, Lead Scoring Module 132, and Predictive Analytics Module 134 allows the Analytics Module to assess the impact of factors such as email subject lines, content relevance, and send times on the campaign's success. Based on this analysis, the physical therapy center can make data-driven adjustments to enhance the performance of future email campaigns.

At step 606, the Analytics Module 122 employs artificial intelligence or machine learning algorithms to generate reports and visualizations, presenting the analyzed data and insights to healthcare practitioners in a comprehensive and easily digestible format. By incorporating data from the Chatbot Module 128, Correlation Module 130, Lead Scoring Module 132, and Predictive Analytics Module 134, the Analytics Module 122 can provide an enhanced understanding of the effectiveness of various marketing campaigns and strategies. In one example, the Analytics Module 122 generates a dashboard displaying the response rate, engagement, and conversion metrics for a myopia doctor's email campaign. By utilizing data from the Chatbot Module 128, the Analytics Module 122 can also present insights into patient interactions and queries, helping the doctor identify common concerns or questions that can be addressed in future email campaigns. Additionally, the integration with the Correlation Module 130 enables the identification of patterns in patient engagement, allowing the doctor to optimize email content, subject lines, and send times for better results. In another example, the Analytics Module 122 creates a report for a dermatology clinic's social media marketing campaign, presenting metrics such as reach, engagement, and conversion rates. By integrating data from the Lead Scoring Module 132, the report can also include information on the quality of leads generated from the campaign, helping the clinic understand which content and strategies are most effective at attracting high-value patients. Furthermore, the connection with the Predictive Analytics Module 134 allows the Analytics Module to provide recommendations on content types, posting times, and audience targeting that are likely to yield better outcomes in future campaigns.

At step 608, the Analytics Module 122 leverages artificial intelligence or machine learning algorithms to identify opportunities for optimization and improvement in marketing campaigns, messaging, and content, based on the analyzed data. By utilizing insights from the Chatbot Module 128, Correlation Module 130, Lead Scoring Module 132, and Predictive Analytics Module 134, the Analytics Module 122 can provide targeted recommendations to enhance marketing effectiveness and patient engagement. In one example, the Analytics Module 122 identifies that a myopia doctor may improve patient engagement by adjusting the frequency or timing of their email campaign. By incorporating data from the Chatbot Module 128, the Analytics Module 122 can also recognize common topics of interest or concern among patients and suggest incorporating these themes into future email content. In addition, with insights from the Correlation Module 130, the Analytics Module can identify patterns in patient responsiveness to different email subject lines or content types, further refining the doctor's email campaign strategy. In another example, the Analytics Module 122 pinpoints opportunities for a dental clinic to optimize their social media marketing campaign, such as adjusting post frequency or experimenting with different content formats. By integrating data from the Lead Scoring Module 132, the Analytics Module can provide insights on the quality of leads generated by the campaign, helping the clinic focus on strategies that attract high-value patients. Additionally, with information from the Predictive Analytics Module 134, the Analytics Module can offer data-driven recommendations on the ideal times to post, audience targeting parameters, and content topics that are more likely to resonate with the target audience.

At step 610, the Analytics Module 122 stores the analyzed data and generated insights within the Healthcare Marketing Automation System 106 for future reference and use, allowing healthcare practitioners to review and compare the performance of future campaigns. The module integrates with Chatbot Module 128, Correlation Module 130, Lead Scoring Module 132, and Predictive Analytics Module 134 to enhance its analysis and provide more targeted recommendations for marketing optimization. In one example, a myopia doctor uses the Analytics Module 122 to analyze the performance of an email campaign. The module saves the campaign data, including response rates and engagement metrics, enabling the practitioner to compare the results with future campaigns. By incorporating insights from the Chatbot Module 128, the doctor can identify topics that resonate with patients and incorporate them into future email content. In another example, a dry eye clinic employs the Analytics Module 122 to evaluate the effectiveness of a marketing campaign featuring educational articles and videos about dry eye syndrome. The module considers patient demographics, clinical data, communication preferences, and engagement with the content. After analysis, the module identifies that patients with moderate dry eye symptoms are more likely to schedule appointments after viewing videos rather than reading articles. The healthcare practice adjusts its marketing strategy accordingly to improve patient outreach. With data from the Correlation Module 130, the clinic can also identify patterns in content engagement and preferences, tailoring their marketing efforts for optimal results.

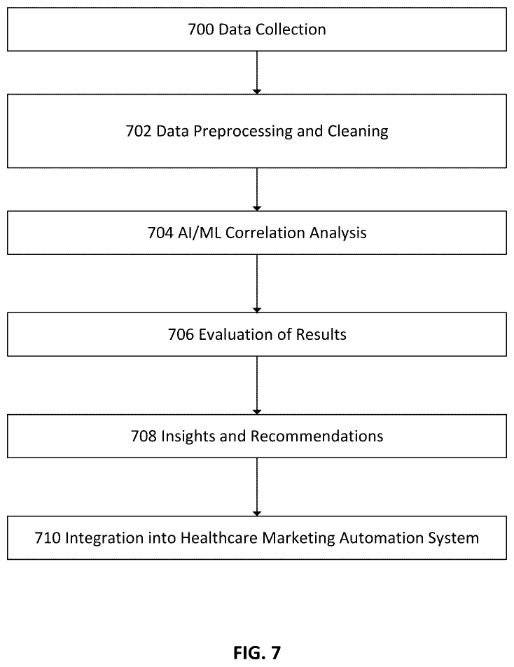

FIG. 7 illustrates a correlation module 130, according to an embodiment. The process begins at step 700, the Correlation Module 130 collects data from various sources, including the Patient Database 124, Analytics Module 122, Chatbot Module 128, Lead Scoring Module 132, and Predictive Analytics Module 134. For example, the module may gather patient demographics, clinical data, engagement metrics, and chatbot interaction data. In one example, the Correlation Module 130 uses clustering algorithms to segment patients based on similar characteristics, such as age, diagnosis, and communication preferences. In another example, the module employs dimensionality reduction techniques, such as Principal Component Analysis (PCA), to analyze and visualize high-dimensional data and identify patterns. At step 702, the Correlation Module 130 preprocesses and cleans the collected data to ensure its quality and consistency. For example, the module may handle missing values, remove outliers, and normalize data. In one example, the Correlation Module 130 uses techniques like data imputation to fill in missing values, improving the reliability of the analysis. In another example, the module applies feature scaling methods, such as Min-Max normalization or Standardization, to ensure all features are on a comparable scale. At step 704, the Correlation Module 130 applies AI/ML techniques to identify correlations, patterns, and trends within the data. For example, the module may use supervised or unsupervised learning algorithms to discover relationships between variables. In one example, the Correlation Module 130 employs association rule mining to identify frequent co-occurrences of patient attributes, treatments, and outcomes, providing insights into effective healthcare marketing strategies. In another example, the module uses regression analysis to model the relationships between patient characteristics, marketing efforts, and patient engagement or conversion rates. In another example, the Correlation Module 130 employs a clustering algorithm, such as k-means, to group patients based on their preferences, diagnoses, and demographic data, providing valuable insights for targeted marketing campaigns. In another example, the module uses a decision tree algorithm to model the relationships between patient characteristics, elements of digital media asset, marketing efforts, and patient engagement or conversion rates, enabling the system to optimize marketing strategies. At step 706, the Correlation Module 130 evaluates the results of the analysis, assessing the significance and reliability of identified correlations. For example, the module may use statistical tests, cross-validation, or model performance metrics to validate the findings. In one example, the Correlation Module 130 applies hypothesis testing, such as t-tests or chi-squared tests, to determine the statistical significance of the identified correlations. In another example, the module uses performance metrics like accuracy, precision, recall, or F1-score to assess the reliability and effectiveness of the AI/ML models. The Correlation Module 130 may apply correlation coefficients, such as Pearson or Spearman, to measure the strength and direction of the relationships between variables. In another example, the module uses performance metrics like mean squared error, R-squared, or adjusted R-squared to assess the goodness-of-fit of the regression models and the reliability of the identified relationships. At step 708, the Correlation Module 130 provides insights and recommendations based on the identified correlations to improve the Healthcare Marketing Automation System 106 and its various modules. The correlation may be between a group of similar patients to a type and channel for delivery of digital media asset. The correlation may be between an area of practice for the practitioner and engagement with a type of digital media asset. For example, the module may suggest adjustments to messaging, content, or targeting strategies based on the discovered patterns. In one example, the Correlation Module 130 recommends that a dermatology practice should target younger patients with quizzes and older patients with surveys based on the identified preferences of different age groups. In another example, the module suggests that a physical therapy clinic should use exercise demonstration videos to target patients with sports-related injuries, as these patients show higher engagement with such content. In another example, the Correlation Module 130 recommends that a myopia doctor should focus on delivering educational content about eye care and prevention measures, as the analysis has revealed a strong correlation between the consumption of such content and patients scheduling appointments. In another example, the module suggests that a mental health and addiction treatment center should adjust the types of content shared with patients at different stages of recovery, as the analysis has identified a correlation between content type and patient engagement at various recovery stages. At step 710, the Correlation Module 130 integrates the derived insights and recommendations into the Healthcare Marketing Automation System 106, enabling the system to adjust its strategies and optimize its performance. In one example, the Correlation Module 130 updates the Lead Scoring Module 132 with new insights about patient preferences, allowing the module to better prioritize and target potential leads. In another example, the module shares the discovered correlations with the Predictive Analytics Module 134, enhancing the module's ability to forecast patient behavior and outcomes.

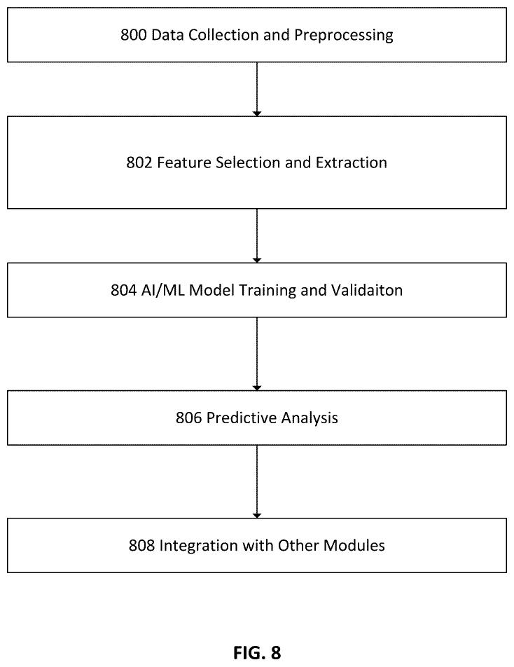

FIG. 8 displays the predictive analytics module 134. The process begins with at step 800, the Predictive Analytics Module 134 collects and preprocesses data from various sources, such as the Patient Database 124 and engagement metrics from marketing campaigns. For example, the module gathers data on a patient's age, gender, diagnosis, treatment history, and interactions with email campaigns. In another example, the module collects data on a specific demographic group's preferences and responses to various types of content and communication channels. At step 802, the Predictive Analytics Module 134 selects and extracts relevant features from the collected data to create a feature set for AI/ML model training. For example, the module may identify variables like diagnosis, treatment plan, and preferred communication channels as important features for predicting a patient's likelihood of scheduling an appointment. In another example, the module may determine that content format and frequency of communication are critical features for predicting engagement levels within a specific demographic group.

At step 804, the Predictive Analytics Module 134 trains and validates AI/ML models using the selected features and historical data. For example, the module may use supervised learning algorithms, such as decision trees or support vector machines, to predict the likelihood of a patient with specific characteristics scheduling an appointment after receiving a personalized email. In another example, the module may employ unsupervised learning algorithms, such as clustering or principal component analysis, to identify patterns in engagement data for a specific demographic group, which can help predict future trends in content preferences and communication channels.

At step 806, the Predictive Analytics Module 134 performs predictive analysis using the trained AI/ML models to forecast behaviors, trends, or engagement metrics. For example, the module may predict that patients diagnosed with a specific condition are more likely to schedule appointments if they receive educational content through their preferred communication channels. In another example, the module may forecast that a specific demographic group will show increased engagement with video content as opposed to text-based content in the next marketing campaign.

At step 808, the Predictive Analytics Module 134 provides the predictive analysis results to other modules within the Healthcare Marketing Automation System 106 for improved patient outcomes and business outcomes. For example, the module may share its predictions with the Content Creation Module 120 to generate targeted educational materials for patients diagnosed with a specific condition. In another example, the module may provide its forecasts to the Analytics Module 122, which can analyze the performance of the marketing campaign featuring video content for a specific demographic group and adjust the strategy accordingly for increased engagement and conversion rates. In one embodiment, the Predictive Analytics Module 134 collects data on patients' treatment history, demographics, and interactions with previous marketing campaigns, and preprocesses this data for analysis. Relevant features, such as diagnosis, preferred communication channels, and response rates to different content formats, are extracted to create a feature set for AI/ML model training. The module employs supervised learning algorithms, such as decision trees, to predict the likelihood of a patient scheduling an appointment after receiving a personalized email containing educational content related to their condition. The trained AI/ML model predicts that patients diagnosed with a specific condition are more likely to schedule appointments if they receive educational content through their preferred communication channels. These predictions are then shared with other modules within the Healthcare Marketing Automation System 106, such as the Content Creation Module 120, which can generate targeted educational materials for patients diagnosed with the specific condition. This integration of predictive analysis allows the Healthcare Marketing Automation System 106 to optimize patient outcomes and improve business performance by tailoring marketing campaigns to individual patient needs and preferences.

In another embodiment, the Predictive Analytics Module 134 gathers data on a specific demographic group's preferences and responses to various types of content and communication channels. By extracting features such as content format and frequency of communication, the module creates a feature set for AI/ML model training. Using unsupervised learning algorithms, such as clustering, the module identifies patterns in engagement data for the specific demographic group, which can help predict future trends in content preferences and communication channels. For instance, the module may forecast that the demographic group will show increased engagement with video content as opposed to text-based content in the next marketing campaign. The Predictive Analytics Module 134 provides these forecasts to the Analytics Module 122, which can analyze the performance of the marketing campaign featuring video content for the specific demographic group and adjust the strategy accordingly for increased engagement and conversion rates. In this manner, the Healthcare Marketing Automation System 106 leverages predictive analysis to optimize both patient outcomes and business performance by adapting marketing strategies to meet the preferences of specific demographic groups.

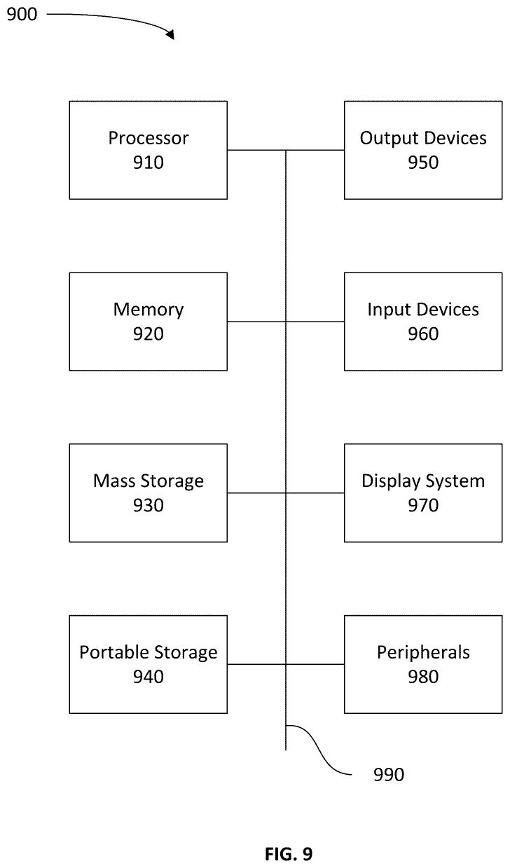

FIG. 9 illustrates an exemplary computing system 900 that may be used to implement an embodiment of the present invention. The computing system 900 of FIG. 9 includes one or more processors 910 and memory 920. Main memory 920 stores, in part, instructions and data for execution by processor 910. Main memory 920 can store the executable code when in operation. The system 900 of FIG. 9 further includes a mass storage device 930, portable storage medium drive(s) 940, output devices 950, user input devices 960, a graphics display 970, and peripheral devices 980.

The components shown in FIG. 9 are depicted as being connected via a single bus 990. However, the components may be connected through one or more data transport means. For example, processor unit 910 and main memory 920 may be connected via a local microprocessor bus, and the mass storage device 930, peripheral device(s) 980, portable storage device 940, and display system 970 may be connected via one or more input/output (I/O) buses.

Mass storage device 930, which may be implemented with a magnetic disk drive or an optical disk drive, is a non-volatile storage device for storing data and instructions for use by processor unit 910. Mass storage device 930 can store the system software for implementing embodiments of the present invention for purposes of loading that software into main memory 920.

Portable storage device 940 operates in conjunction with a portable non-volatile storage medium, such as a floppy disk, compact disk or Digital video disc, to input and output data and code to and from the computer system 900 of FIG. 9. The system software for implementing embodiments of the present invention may be stored on such a portable medium and input to the computer system 900 via the portable storage device 940.

Input devices 960 provide a portion of a user interface. Input devices 960 may include an alpha-numeric keypad, such as a keyboard, for inputting alpha-numeric and other information, or a pointing device, such as a mouse, a trackball, stylus, or cursor direction keys. Additionally, the system 900 as shown in FIG. 9 includes output devices 950. Examples of suitable output devices include speakers, printers, network interfaces, and monitors.

Display system 970 may include a liquid crystal display (LCD) or other suitable display device. Display system 970 receives textual and graphical information, and processes the information for output to the display device.

Peripherals 980 may include any type of computer support device to add additional functionality to the computer system. For example, peripheral device(s) 980 may include a modem or a router.

The components contained in the computer system 900 of FIG. 9 are those typically found in computer systems that may be suitable for use with embodiments of the present invention and are intended to represent a broad category of such computer components that are well known in the art. Thus, the computer system 900 of FIG. 9 can be a personal computer, hand held computing device, telephone, mobile computing device, workstation, server, minicomputer, mainframe computer, or any other computing device. The computer can also include different bus configurations, networked platforms, multi-processor platforms, etc. Various operating systems can be used including Unix, Linux, Windows, Macintosh OS, Palm OS, and other suitable operating systems.

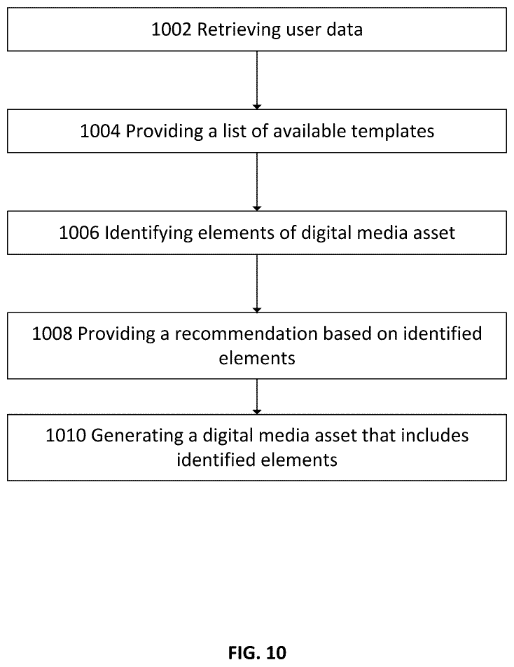

FIG. 10 is a flow chart illustrating an exemplary method for delivery of personalized user content. At step 1002, user data from a plurality of users are retrieved from Patient Database 124. The user data may include patient demographics data, such as patient age, gender, and race, patient diagnosis, stage of the healthcare journey, treatment history, previous health condition, current health condition, previous engagement history with digital media assets by the patients, history of interaction with Chatbot Module 128, patient preferences, etc. The user data may be retrieved based on a request received from Base Module 108, User Module 110 or the Marketing Automation Module 116. The request may indicate that a practitioner would like to access the patient data or generate a certain type of content to deliver to the patients. The users may be grouped based on one or more factors, such as sharing a common characteristic in patient demographics or health history. For example, the Correlation Module 130 may employ a clustering algorithm, such as k-means, to group patients based on their preferences, diagnoses, and demographic data.

At step 1004, Content Generation Module 120 may utilize AI and machine learning algorithms to identify and provide a list of available content templates for generating a digital media asset based on the content type that the practitioner would like to deliver to the users. Content Generation Module 120 may further provide other resources and tools related to the content type.

At step 1006, one or more elements of digital media assets may be identified based on the one or more factors. Correlation Module 130 may use a decision tree algorithm to model the relationships between patient characteristics, elements of digital media asset, marketing efforts, and patient engagement or conversion rates. For example, Correlation Module 130 may apply machine learning techniques to find a strength of correlation between the common characteristic and an element of digital media asset that may be effective for the users with the characteristic. The strength of correlation may be converted to and assigned a lead score, which predicts the likelihood of a prospective patient engagement with the digital media asset. The lead score may be based on historical engagement by the group of users or a user in the group with other digital media assets having a certain element in the content in common with the digital media asset to be generated. The lead score may be calculated by the Lead Scoring Module 132. The elements of the digital media content may include a specific video style background music, a type of message, etc. Prospective engagement may include patient interacting with the digital media asset, scheduling an appointment, or undergoing treatments.

At step 1008, a recommendation may be generated regarding adjusting content of the templates based on the identified elements. The recommendation may be generated by the Content Generation Module 120 and provided to Practitioner Device 102 via User Module 110 or Display Module 112. The elements of digital media that is correlated with a high predicted engagement with the group of users may be included or added whereas the elements in the template that is correlated with a low predicted engagement with the group of users may be excluded. The recommendation may further include a type of communication channel or a frequency of delivery of digital media asset that is associated with a high engagement with the group of users.

At step 1010, the Content Generation Module 120 may adjust the content of the templates to generate a customized content based on the selection from the recommended elements of the digital media asset. The adjusted digital media asset may be generated by the Content Generation Module 120 and provided to Practitioner Device 102 or Patient Devices 104 via User Module 110, Display Module 112, Messaging Module, 118, or Patient Module 126.

The actual engagement with the delivered digital media asset may be tracked and used to update the metrics associated with calculation of the lead score of the group of users.

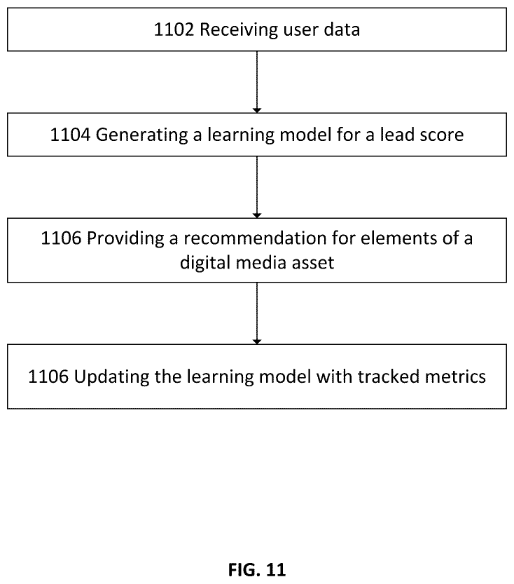

FIG. 11 is a flow chart illustrating an exemplary method for delivery of personalized user content. At step 1102, user data from a plurality of users are received from Patient Database 124. The user data may include patient demographics data, such as patient age, gender, and race, patient diagnosis, stage of the healthcare journey, treatment history, previous health condition, current health condition, previous engagement history with digital media assets by the patients, history of interaction with Chatbot Module 128, patient preferences, etc.

At step 1104, the received data is used to generate a learning model for a lead score. The lead score is associated with a predicted engagement. The lead score may be calculated by the Lead Scoring Module 132 The lead score may be based on demographics, medical history, engagement with digital media assets, and interactions with the chatbot. Patient engagement may include patient interacting with the digital media asset, scheduling an appointment, or undergoing treatments.

At step 1106, a recommendation is provided for elements of digital media asset based on a correlation between each element and a likelihood of engagement. The elements of the digital media content may include a specific video style background music, a type of message, etc. Correlation Module 130 may use a decision tree algorithm to model the relationships between patient characteristics, elements of digital media asset, marketing efforts, and patient engagement or conversion rates. The recommendation may be generated by the Content Generation Module 120 and provided to Practitioner Device 102 via User Module 110 or Display Module 112. The recommendation may include elements of digital media asset with a high likelihood of predicted engagement with the users. The recommendation may further include a type of communication channel or a frequency of delivery of digital media asset that is associated with a high engagement with the users.

At step 1108, one or more metrics associated with the digital media asset are tracked. The metrics are associated with engagement with the digital media asset, which may include key performance indicators, such as clickthrough, view count, response rate (replies), conversion (scheduled appointments), retention (predicted vs. actual milestone on healthcare journey), payments, referrals, etc. The tracked metrics, which indicate actual engagement with the digital media asset provided to a user, are used to update the learning model for the lead score.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

What is claimed is:

1. A computer-implemented method for generation of personalized user content, the method comprising:
receiving an indication regarding a selected type of media content corresponding to an identified content template among a plurality of different available content templates;
identifying one or more content elements that are associated with one or more user factors correlating to a lead score of a group of patients;
identifying one or more content adjustments to the selected content template based on the identified elements; and
generating customized content by adjusting the template in accordance with the identified adjustments, wherein the customized content includes the identified content elements.

2. The method of claim 1, further comprising storing the plurality of different available content templates in memory, each content template corresponding to a different type of media content.

3. The method of claim 1, further comprising storing data in memory regarding a plurality of patients, wherein each patient is grouped based on one or more of the factors.

4. The method of claim 3, further comprising retrieving the data from one or more devices over a communication network.

5. The method of claim 1, further comprising generating a recommendation regarding the identified adjustments to the template.

6. The method of claim 1, wherein the factors include demographics and health history of the users.

7. The method of claim 1, wherein the lead score is associated with a history of engagement by the grouped users.

8. The method of claim 1, further comprising providing a recommendation regarding delivery of the customized content.

9. The method of claim 8, wherein the recommendation regarding the delivery includes a preferred communication channel.

10. The method of claim 8, wherein the recommendation regarding the delivery includes a frequency of delivery.

11. The method of claim 1, further comprising tracking engagement with the customized content, wherein the lead score associated with the grouped patient is updated based on the tracked engagement.

12. A computer-implemented method for modeling engagement with customized content, the method comprising:
receiving data regarding a plurality of user factors associated with a plurality of different users engaging with different digital media assets;
generating a learning model for a group of the users based on the received data, wherein the learning model correlates one or more of the content elements to a level of engagement by the group;
analyzing a digital media asset based on the learning model to identify one or more content elements present in the digital media asset;
generating a recommendation regarding a set of one or more adjustments to the identified elements based on a correlation between the adjusted elements and a predicted level of engagement; and
tracking one or more metrics associated with the digital media asset, wherein the learning model is updated based on the tracked metrics.

13. The method of claim 12, further comprising defining the group of users in accordance with one or more user factors.

14. The method of claim 13, wherein the user factors include one or more of demographics, medical history, and engagement history.

15. The method of claim 12, wherein the engagement history includes engagement with a chatbot.

16. The method of claim 12, further comprising generating a different learning model for a different group of the users associated with different user factors.

17. The method of claim 16, wherein analyzing the digital media asset based on the different learning model results in a different recommendation for a different set of adjustments.
